(19) United States
(12) Patent Application Publication
(10) Pub. No.: US 2003/0088363 A1
(43) Pub. Date: May 8, 2003

(54) Encoding characteristics of a biological sample

(76) Inventor: David Benjamin Aronow, Amherst, MA (US)

Correspondence Address: Patent Group, Foley, Hoag & Eliot LLP, One Post Office Square, Boston, MA, 02109-2170, US

(21) Appl. No.: 10/053,082

(22) Filed: Nov. 2, 2001

(51) Int. Cl. G06F 19/00; G01N 33/48; G01N 33/50; G06G 7/48; G06G 7/58

(52) U.S. Cl. 702/19; 700/1; 703/11

(57) ABSTRACT

In general, in one aspect, the disclosure describes a method of encoding a characteristic of a sample. The method includes identifying a collection of more than one codes of a standard coding scheme where different codes correspond to different standard coding scheme concepts. The method forms a pre-coordinated code not found in the standard coding scheme from a concatenation of the codes. The method also stores the pre-coordinated code along with other pre-coordinated codes.

### BACKGROUND

A wide variety of standard coding schemes enable medical professionals to compactly encode a diagnosis. For example, a standard coding scheme known as ICD (International Classification of Diseases) includes a code of “564.2” for a diagnosis of “post gastric surgery syndrome”. Encoding a diagnosis using this code reduces the amount of computer storage needed to store a diagnosis and can ease data retrieval and other computer automation tasks.

Another standard coding scheme is known as SNOMED (Systemized Nomenclature of Human and Veterinary Medicine). SNOMED provides codes that represent different concepts. For example, the SNOMED code “126824007” represents the concept “Neoplasm of stomach”. This concept is an example of a disease concept. In addition to disease concepts, SNOMED provides other categories (“axes”) of concepts such as concepts expressing topology (e.g., body parts and regions), etiology (e.g., the causes of a disease), morphology (e.g., exhibited changes), and procedure (e.g., the administrative, preventive, diagnostic, and therapeutic actions taken to prevent or cure a disease, illness, or injury).

To describe a condition, a medical professional can combine SNOMED concepts from different axes. The SNOMED scheme provides flexibility and enables a medical professional to freely combine different codes in a wide variety of combinations to describe a diagnosis to their liking.

### SUMMARY

In general, in one aspect, the disclosure describes a method of encoding a characteristic of a biological sample. The method includes identifying a collection of more than one codes of a standard coding scheme where different codes correspond to different standard coding scheme concepts. The method forms a pre-coordinated code not found in the standard coding scheme from a concatenation of the codes. The method also stores the pre-coordinated code along with other pre-coordinated codes.

Embodiments may include one or more of the following features. The method may include storing a collection of one or more lexical terms describing a concept associated with the pre-coordinated code. The codes may be SNOMED (Systemized Nomenclature of Human and Veterinary Medicine) codes. Concatenating the codes may conform to at least one syntax rule such as a rule specifying an ordering of terms according to their SNOMED axis.

The method may further include providing the stored pre-coordinated codes for assignment to a biological sample. The sample may be an excess tissue sample received from a donor institution. Providing the pre-coordinated codes for assignment may include displaying at least one selection menu including lexical terms of the concepts associated with the pre-coordinated codes. Providing the stored pre-coordinated codes for assignment may include providing the stored codes as elements of a set of user interface instructions (e.g., markup language instructions) transmitted over a network.

The method may further include receiving a query identifying one of the pre-coordinated codes and identifying a collection of samples. The query may be received via a computer network, for example, encoded within a network transfer protocol message.

Forming the pre-coordinated code may include concatenation of the more than one codes with code separating delimiters. The pre-coordinated code may correspond to a diagnosis concept, a tissue concept, or a procedure concept.

In general, in one aspect, the disclosure describes a computer program product, disposed on a computer readable medium, for encoding a characteristic of a biological sample. The program includes instructions for causing a processor to identify a collection of more than one codes of a standard coding scheme where different codes correspond to different concepts of the standard coding scheme. The program also includes instructions that form a pre-coordinated code from a concatenation of the more than one codes, the pre-coordinated code not being found in the standard coding scheme. The program also includes instructions that store the pre-coordinated code along with other pre-coordinated codes.

Advantages will become apparent in view of the following description, including the figures and the claims.

### BRIEF DESCRIPTION OF THE DRAWINGS

FIGS. 1 to 3 illustrate a system for making excess tissue samples available to researchers.

FIG. 4 illustrates formation of a highly specific pre-coordinated code from a concatenation of standard, less specific coding scheme codes.

FIGS. 5 and 6 illustrate user interfaces for generating a query for samples meeting query criteria.

FIG. 7 is a flowchart of a process for generating and using pre-coordinated codes to identify samples meeting a query criteria.

### DETAILED DESCRIPTION

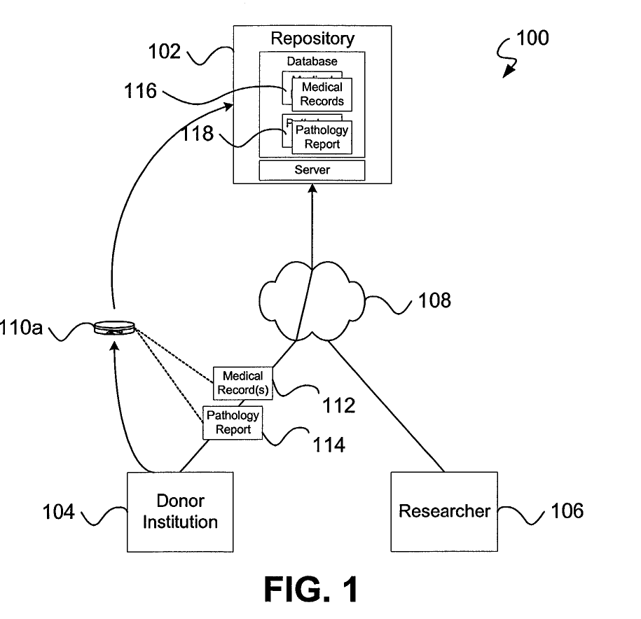

FIGS. 1 to 3 illustrate a system 100 that can enable researchers 106 to use otherwise discarded biological samples 110a in their studies. Such samples can include tissue samples and/or samples of blood or other bodily fluids. As shown in FIG. 1, the system 100 includes a donor institution 104 with a pathology department. Often such departments use only a portion of a sample to perform a given medical test. In the past, the remainder was typically discarded despite the difficulty of obtaining such samples for research.

As shown in FIG. 1, a repository 102 collects excess samples 111a from a donor institution 104 for distribution to interested researchers 106. In addition to physically delivering the samples 110a to the repository, a donor institution 104 can also transmit information specifying characteristics of a sample that may be of interest to a researcher 106. For example, as shown, the institution 104 transmits a medical record 112 of a patient providing the sample as well as a pathology report 114 about the sample. The transmission may be performed via interaction with a repository 102 server over a network 108 using a variety of network transfer protocols such as HTTP (HyperText Transfer Protocol) or FTP (File Transfer Protocol).

The medical report 112 may include physical data (e.g., the patient's approximate age, weight, gender) and health data such as different health risks (e.g., whether the patient smokes cigarettes) and/or diagnosed illness(es) of the patient. The medical report 112 may also include demographic data. For confidentiality, the medical report 112 may omit the patient's real name and other personal identifiers.

The pathology report 114 includes the results of the donor institution's 104 analysis of the sample 110a. For example, the pathology report 114 may include data identifying the sample 110a as cancerous. The pathology report 114 may also include data about the sample such as where a tissue sample was extracted from and other sample characteristics.

As shown in FIG. 1, the donor institution 104 can transmit the reports 112, 114 over a network 108, such as the Internet, to a repository 102. The repository 102 can store the received records 116, 118 associated with the sample 110a in a database that stores records associated with other samples.

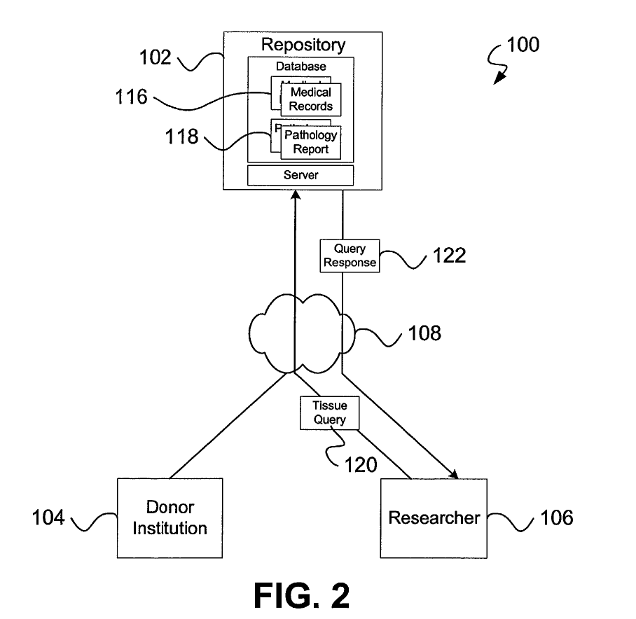

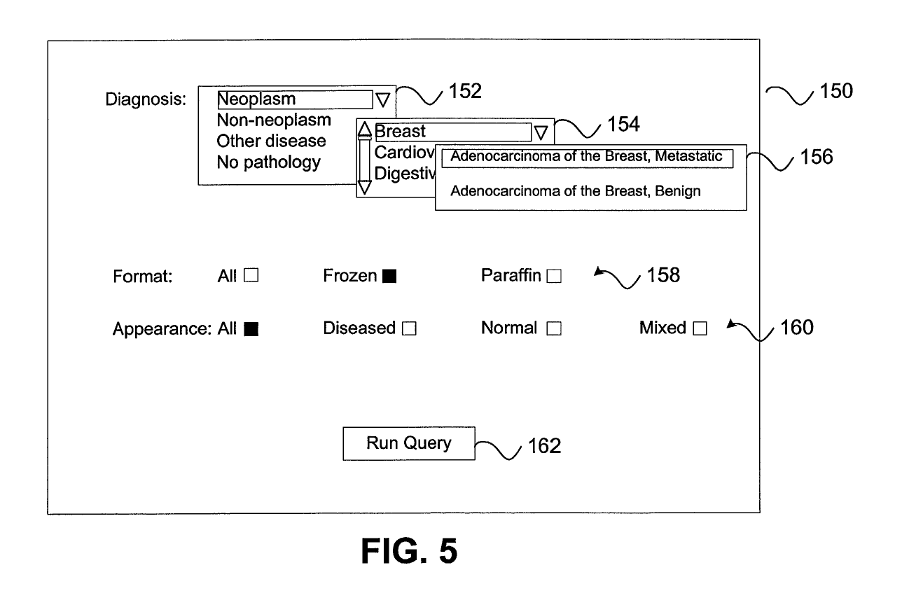

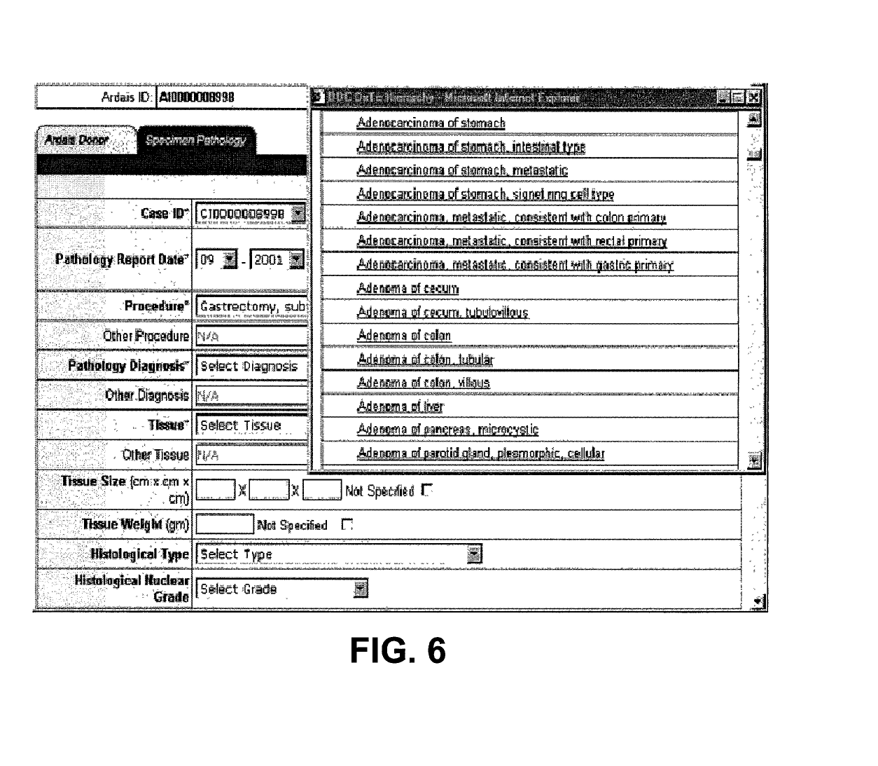

As shown in FIG. 2, the repository 102 server can permit researchers 106 to browse an on-line inventory for samples 110 meeting specified criteria. As shown, a researcher 106 can submit a query 120 to the server 102 over the network 108 specifying criteria, for example, of the tissue type, diagnosis, and so forth. For instance, the researcher 106 may interact with a user interface such as the one shown in FIG. 5 or 6. After identifying samples 110 matching the query 120, the repository 102 server can transmit a query response 122 back to the researcher 106. For example, the response 122 may feature a dynamically constructed web-page that includes a list of available tissue samples 110. Such a web-page may include links featuring images of the tissue samples, the “clinical story” of the donors, or other information in the pathology or medical reports.

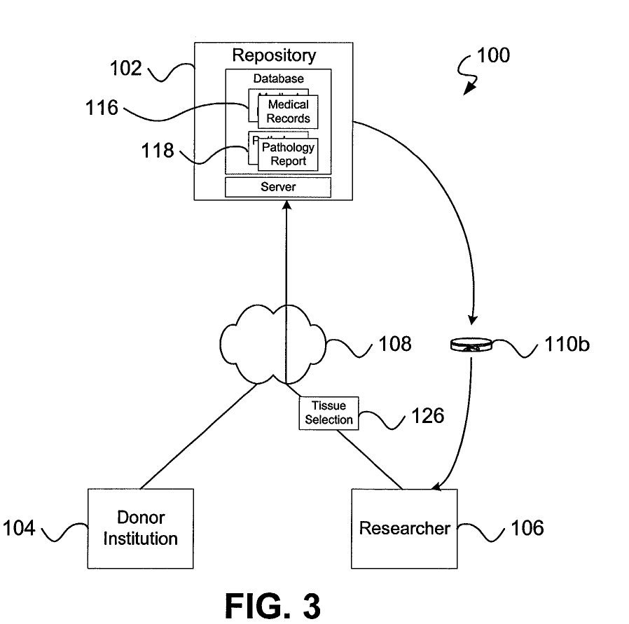

As shown in FIG. 3, after receiving a selection 126 of desired tissue samples from the researcher 106, the repository server 102 can initiate physical delivery 110b of the sample in a wide variety of researcher specified forms. For example, the researcher 106 can request frozen or formalin fixed gross samples, frozen or paraffin tissue blocks, extracted RNA, DNA and proteins, tissue microarrays, RNA derived from Laser Capture Microdissection, and so forth. The research may also request images of selected samples.

While FIGS. 1 to 3 show a single donor institution 104 and researcher 106, the system 100 can provide services to many different institutions 104 and researchers 106.

The diverse needs of users, the variety of concepts embodied by a given sample, and the flexibility of many coding systems can make searching for a sample having particular characteristics more difficult. Thus, the system shown in FIGS. 1 to 3 can benefit from a scheme that enables users to specify tissue samples of interest with great specificity.

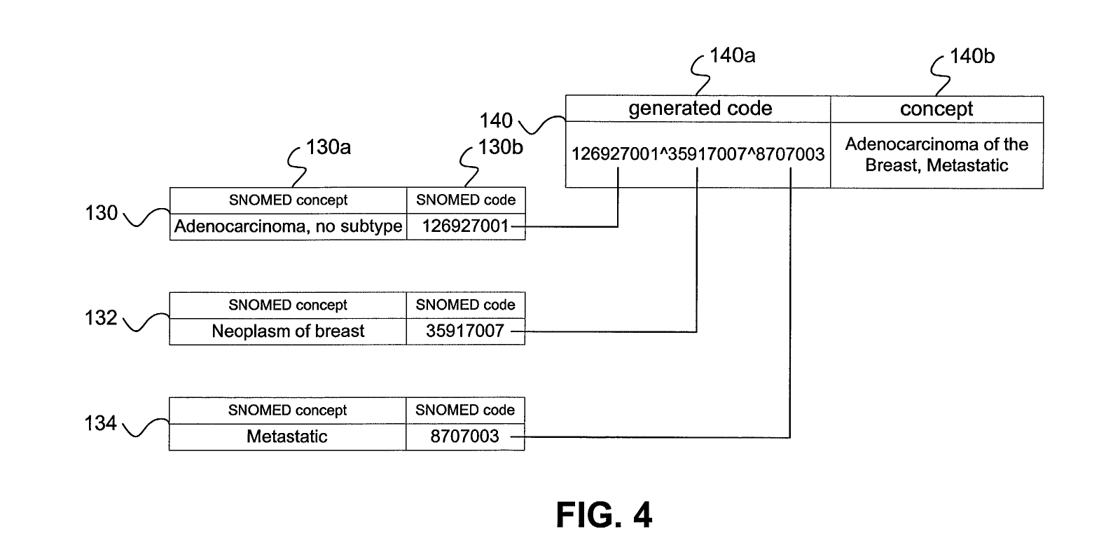

FIG. 4 illustrates a coding approach that uses SNOMED-RT (SNOMED-Reference Terminology) concepts as building blocks for concepts of greater specificity than provided by existing SNOMED-RT concepts. That is, a finely grained concept can be represented through the pre-coordination of a collection of SNOMED concept codes. For example, SNOMED does not currently offer a code for “metastatic adenocarcinoma of the stomach”. However, a pre-coordinated code for this concept may be fashioned from the combination of SNOMED concept codes for “neoplasm of stomach” (126824007), “adenocarcinoma no subtype” (35917007), and “metastatic” (8707003). That is, combining the codes for these concepts (e.g., “126824007ˆ 35917007ˆ 8707003”) yields a pre-coordinated code for the narrow concept of “metastatic adenocarcinoma of the stomach”. By defining this concept and its associated pre-coordinated code, researchers can narrowly specify concepts and more precisely code information. This can permit more exact categorization and searching. Additionally, preservation of the “building block” codes preserves a broad search capability.

The approach can also “normalize” diagnosis coding across different institutions and researchers. That is, a normalized controlled vocabulary provides explicit, concise, and predictable names across donor institutions and clients, for both data entry and query parameter setting. Thus, the approach can ensure that different institutions encode the same diagnosis using the same code. This can also enable researchers to find tissue samples of interest without the guesswork involved in hypothesizing how others may have encoded a diagnosis.

In greater detail, FIG. 4 illustrates a collection 130-134 of concepts 130a and their corresponding codes 130b. While illustrated using SNOMED concepts and codes, a wide variety of other standard coding schemes may be used.

As shown, the different codes in the collection 130-134 correspond to different related concepts regarding a diagnosis. In the case of SNOMED, the collection 130-134 of codes may feature codes from different SNOMED axes (categories). As shown, concatenating the collection 130-134 of codes together can create a pre-coordinated code 140a. The pre-coordinated code 140a corresponds to a textual description of a narrow concept 140h. Appendix A includes a sample database of such codes expressing a wide variety of diagnoses. Again, institutions can use these codes to encode their diagnoses of submitted samples. Similarly, researchers can use these codes to search for samples of interest.

As shown in FIG. 4, the concatenation may feature delimiters separating the different codes (e.g., the “ˆ ” characters). Alternatively, the code 140a may pad each “sub-code” 130b such that each concept code occupies a predetermined portion of the resulting code 140a if desired. Regardless, the code 140a retains the information of contributing codes 130-134 in the pre-coordinated code 140a.

While straightforward, this technique can provide a number of benefits. Again, adopting the technique can permit researchers to identify characteristics with great specificity. The approach can also result in the presentation of a single way to encode a diagnosis associated with a tissue sample. Again, this can ease retrieval by researchers.

While the resulting code 140a does not appear in the standard coding scheme of the contributing codes 130-134 (e.g., the concatenated code does not appear in SNOMED), the code 140a construction preserves the constituent standard code building blocks. This can enable researchers to perform a search for tissue samples based on a standard SNOMED code as well as the pre-coordinated code 140a. For example, a researcher can search for all tissue samples that include the SNOMED code, “8707003”, for the concept “Metastatic”. Though tissue samples may have been assigned a composite code 140a of “126927001ˆ 35917007ˆ 8707003”, the retrieval software can identify all samples featuring the SNOMED code “8707003” within their pre-coordinated codes.

While FIG. 4 illustrates an example of a pre-coordinated concept formed from three “sub-concepts”, different pre-coordinated codes may feature different number of concepts. For example, a concept of “adenocarcinoma of stomach, signet ring cell type” may be formed from the concepts “neoplasm of stomach” and “signet ring cell carcinoma”, represented by codes 126824007 and 87737001, respectively.

The concatenation order of codes and the expression of a concept may be performed in accordance with different syntax rules. For example, a rule may specify an ordering of terms according to their SNOMED axis (e.g., SNOMED Disease or Disorder code, Morphology code, Site or Type code, followed by Modifying codes). Such a general rule may be qualified. For example, a rule may remove terms (e.g., “invasive”, “infiltrative”, “residual”, and “minimal”) from a concept name or replace terms (e.g., use “focal” instead of “multifocal” if the latter is specified) to enhance normalization.

A given coding scheme may sometimes fail to offer a useful building block for inclusion in a pre-coordinated code/concept. For example, SNOMED-RT does not currently provide a concept or code for the presence of the “BRCA1” gene. Thus, the code manager may create local extensions to a given coding standard. For example, a code manager may define a code of “CA079954” to represent the concept of the “BRCA1” gene, where “CA” identifies a code as an extension to the standard coding scheme.

While described above as creating a pre-coordinated diagnosis code, the techniques may be used to create other kinds of pre-coordinated codes that may describe a characteristic of a sample. For example, the approach described above can be used to create pre-coordinated tissue codes (e.g., codes describing a particular tissue) or procedure codes (e.g., codes describing a particular procedure). For example, a pre-coordinated code for the tissue concept “Tendon of Foot” may be formed by concatenating 13024001 and 56459004 which correspond to the concepts “tendons” and “foot”, respectively.

A system using the pre-coordinated codes may shield end users from explicitly specifying the codes. For instance, FIGS. 5 and 6 illustrate user interfaces that enable a user to specify characteristics of a sample using the pre-coordinated codes. For example, FIG. 5 illustrates a user interface 150 that enables a user to specify a diagnosis by navigating through a series of text description menus 152-156. The menus shown enable the user to select whether or not the diagnosis is neoplastic 152 and the type of tissue sample 154. Based on these narrowing categories, the user interface 150 can present a text menu 156 of specific concepts having pre-coordinated codes.

The user interface 150 may also include other “widgets”. For example, the interface 150 may enable a researcher to specify a format 158 and/or a tissue appearance 160. After selecting a diagnosis 156 and specifying other attributes 158, 160, a user may submit a corresponding query 162. Such a query may include the actual pre-coordinated code corresponding to a selected concept or may include information used to deduce the code such as the concept text or an integer identifying the code. The query may be included as URL parameters or included in a message sent to the repository 102 server.

The user interface shown in FIG. 5 may be expressed in instructions for transmission over the Internet to a users client (e.g., web-browser). For example, the instructions may feature markup language instructions such as HTML (HyperText Markup Language), XML (extensible Markup Language), or another SGML (Standard Generalized Markup Language) language. Such instructions may be transmitted via a network protocol such as HTTP (HyperText Transfer Protocol) and/or HTTPS (HyperText Transfer Protocol Secure).

The user interface of FIG. 5 presents the concept text of pre-coordinated codes to guide a user's selection. However, other implementations may use other user interface techniques. For example, the user interface may present images of tissue samples and user selection of the image to identify a search for like samples. Behind the scenes, the system can identify the each image may have one or more associated pre-coordinated codes for use in the query.

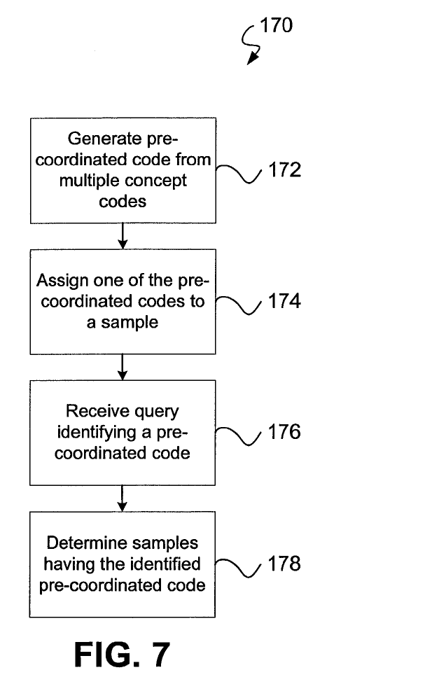

FIG. 7 illustrates operation of a system 170 using the pre-coordinated codes described above. As shown, after generating 172 a pre-coordinated code to represent a concept, the system 170 makes such code available for assignment 174 to a tissue sample, for example, by a donor institution. Thereafter, a received 176 query may identify one of the composite codes. Based on this query, the system 170 can determine tissue samples meeting the user specified criteria.

The techniques described herein are not limited to any particular configuration. For example, the repository 102 may feature an Websphere™ web-server and an Oracle database back-end or some other configuration.

Preferably, the techniques are implemented in computer programs executing on programmable computers that each include a processor, a storage medium readable by the processor (including volatile and non-volatile memory and/or storage elements), at least one input device, and one or more output devices.

Each program is preferably implemented in high level procedural or object oriented programming language to communicate with a computer system. However, the programs can be implemented in assembly or machine language, if desired. In any case the language may be compiled or interpreted language.

Each such computer program is preferably stored on a storage medium or device (e.g., CD-ROM, hard disk, or magnetic disk) that is readable by a general or special purpose programmable computer for configuring and operating the computer when the storage medium or device is read by the computer to perform the procedures described herein. The system may also be considered to be implemented as a computer-readable storage medium, configured with a computer program, where the storage medium so configured causes a computer to operate in a specific and predefined manner.

Other embodiments are within the scope of the following claims.

[image]

[image]

[image]

[image]

[image]

[image]

[image]

[image]

[image]

[image]

[image]

[image]

[image]

What is claimed is:

1. A method of encoding a characteristic of a biological sample, the method comprising:
identifying a collection of more than one codes of a standard coding scheme, different codes corresponding to different concepts of the standard coding scheme;
forming a pre-coordinated code from a concatenation of the more than one codes, the pre-coordinated code not being found in the standard coding scheme; and
storing the pre-coordinated code along with other pre-coordinated codes.

2. The method of claim 1, further comprising storing a collection of one or more lexical terms describing a concept associated with the pre-coordinated code.

3. The method of claim 1, wherein the codes comprise SNOMED (Systemized Nomenclature of Human and Veterinary Medicine) codes.

4. The method of claim 1, wherein concatenating the codes comprises concatenating the codes in accordance with at least one syntax rule.

5. The method of claim 4, wherein the at least one syntax rule comprises a rule specifying an ordering of terms according to their SNOMED axis.

6. The method of claim 1, further comprising providing the stored pre-coordinated codes for assignment to a sample.

7. The method of claim 6, wherein the sample comprises an excess tissue sample received from a donor institution.

8. The method of claim 6, wherein providing the stored pre-coordinated codes for assignment comprises displaying at least one selection menu including lexical terms of concepts associated with the pre-coordinated codes.

9. The method of claim 6, wherein providing the stored pre-coordinated codes for assignment comprises providing the stored codes as elements of a set of user interface instructions transmitted over a network.

10. The method of claim 9, wherein the user interface instructions comprise markup language instructions.

11. The method of claim 6, further comprising
receiving a query identifying a pre-coordinated code; and
identifying a collection of at least one sample having been assigned the pre-coordinated code.

12. The method of claim 11, wherein receiving the query comprises receiving the query via computer network.

13. The method of claim 12, wherein receiving the query comprises receiving the query encoded within a network transfer protocol message.

14. The method of claim 1, wherein forming the pre-coordinated code from a concatenation of the more than one codes comprises concatenation of the more than one codes with code separating delimiters.

15. The method of claim 1, wherein the pre-coordinated code corresponds to a diagnosis concept.

16. The method of claim 1, wherein the pre-coordinated code corresponds to at least one of the following: a tissue concept and a procedure concept.

17. The method of claim 1, wherein at least one of the collection of codes comprises a code not in the standard coding scheme.

18. A computer program product, disposed on a computer readable medium, for encoding a characteristic of a biological sample, the computer program including instructions for causing a processor to:
identify a collection of more than one codes of a standard coding scheme, different codes corresponding to different concepts of the standard coding scheme;
form a pre-coordinated code from a concatenation of the more than one codes, the pre-coordinated code not being found in the standard coding scheme; and
store the pre-coordinated code along with other pre-coordinated codes.

19. The computer program of claim 18, further comprising instructions for causing the processor to store a collection of one or more lexical terms describing a concept associated with the pre-coordinated code.

20. The computer program of claim 18, wherein the codes comprise SNOMED (Systemized Nomenclature of Human and Veterinary Medicine) codes.

21. The computer program of claim 18, wherein the instructions that concatenate the codes comprise instructions that concatenate the codes in accordance with at least one syntax rule.

22. The computer program of claim 21, wherein the at least one syntax rule comprises a rule specifying an ordering of terms according to their SNOMED axis.

23. The computer program of claim 18, further comprising instructions for causing the processor to provide the stored pre-coordinated codes for assignment to a sample.

24. The computer program of claim 23, wherein the instructions that provide the stored pre-coordinated codes for assignment comprise instructions for displaying at least one selection menu including lexical terms of concepts associated with the pre-coordinated codes.

25. The computer program of claim 23, wherein the instructions that provide the stored pre-coordinated codes for assignment comprise instructions that provide the stored codes as elements of a set of user interface instructions transmitted over a network.

26. The computer program of claim 25, wherein the user interface instructions comprise markup language instructions.

27. The computer program of claim 18, further comprising instructions for causing the processor to:
receive a query identifying a pre-coordinated code; and
identify a collection of at least one sample having been assigned the pre-coordinated code.

28. The computer program of claim 27, wherein the instructions that receive the query comprise instructions that receive the query via computer network.

29. The computer program of claim 18, wherein the instructions that form the pre-coordinated code from a concatenation of the more than one codes comprise instructions that concatenate more than one codes with code separating delimiters.

30. The computer program of claim 18, wherein one of the collection of codes comprises a code not in the standard coding scheme.
